(19) United States
(12) Patent Application Publication
(10) Pub. No.: US 2022/0304980 A1
(43) Pub. Date: Sep. 29, 2022

(54) METERED DOSING COMPOSITIONS AND METHODS OF USE OF PSYCHEDELIC COMPOUNDS

(71) Applicant: SW Holdings, Inc, Springfield, OR (US)

(72) Inventors: Craig Michael Arnold, Springfield, OR (US); Parag Bhatt, Springfield, OR (US); Scott Slay, Springfield, OR (US); Michael S Hartman, Milbrae, CA (US)

(21) Appl. No.: 17/624,377

(22) PCT Filed: Jul. 3, 2020
(86) PCT No.: PCT/US2020/040826

§ 371 Date: Jan. 3, 2022

Related U.S. Application Data
(60) Provisional application No. 62/870,722, filed on Jul. 4, 2019.

(51) Int. Cl. A61K 31/4045; A61K 31/137; A61K 31/48; A61K 36/06; A61K 36/33; A61K 36/24; A61K 9/00

(52) U.S. Cl. CPC A61K 31/4045; A61K 31/137; A61K 31/48; A61K 36/06; A61K 36/33; A61K 36/24; A61K 9/008; A61K 9/0078

(57) ABSTRACT

New metered-dosing compositions and methods of use comprising at least one psychedelic compound derived from living organisms such as plants and fungi, synthetically-derived forms or synthetic analogue. The composition for use in metered-dosing administration modalities, including a nasal spray, pressurized metered-dose inhaler, etc.

### CROSS-REFERENCE TO RELATED APPLICATIONS

This application claims priority U.S. Patent Provisional Application No. 62/870,722, “Metered Dosing Compositions of plant and fungal compounds for oral, nasal, sublingual, and topical use” filed on Jul. 4, 2019.

### BACKGROUND

### Field of Use

This disclosure relates to metered dosing compositions and methods of use. The compositions will comprise a compound derived from plants or fungi or their synthetic equivalents.

### Background of Prior Art

Naturally- or biologically-derived compounds from a plant, fungi or other organisms and their synthetic equivalents have been known to be effective in treating or alleviating various psychological and physiological conditions. Many plants and fungi contain compounds that have therapeutic effects on the brain and body. People have consumed these plants and fungi in their raw form and achieved these therapeutic effects. However, consuming the raw form of certain plants and fungi can produce negative side effects, such as uncontrolled and often intense experiences as well as negative digestive effects. Dosing is variable in the raw form and the raw form can be difficult for the body to process. Due to the unknown concentrations of these beneficial compounds when consuming plants and fungi in their raw state, many consumers are apprehensive towards ingesting them. People ingest natural and synthetically derived compounds for various conditions and ailments. Mental and physiological conditions are most commonly treated with doctor-prescribed drugs, over-the-counter supplements, therapies, or a combination of all of the above. Many prescription drugs have several negative physiological side effects on the brain and body as well as addictive or habit forming properties and overuse toxicity that can often overshadow the therapeutic effect of the drug itself. Some current drug regimes or protocols pose the risk of abuse and overuse. In contrast, one of the major highlights of psilocybin and other naturally-derived psychedelic use is that it has not been proven to be habit forming. Additionally, while it can be taken for a psychedelic-type, altered-state-of-consciousness effect, no overuse potential has been reported to this date.

With all drugs or supplements, whether naturally or synthetically derived, there is typically a suggested or prescribed dosage to achieve the desired result. Controlled doses are nearly impossible to achieve with a compound that is in raw or whole form, especially with fungal and plant-based therapies. In raw or natural form there is batch-to-batch variability and is often limited to only oral ingestion, wherein the taste itself can be unpleasant.

Prescription drugs and most supplements are formulated for exact dosing. Current prescription drugs and treatment therapies for increased energy, mental focus, and performance have significant side effects and strong addictive properties. Plant, fungal, and other organically-based supplements are showing a major rise in use and yield increased energy, mental focus, work performance, and physical performance without the side effects and addictive properties of pharmaceutical approaches.

One of the main hurdles to overcome for people being able to safely and effectively utilize these plant-, fungal-, and other biologically-derived compounds and supplements is the availability of a supplement with a known and metered dosage. Currently, no compositions or products exist for metered dosing of compounds containing but not limited to the following (hereinafter “psychedelic compounds”):
1. Psilocybin (O-phosphoryl-4-hydroxy-N,N-dimethyltryptamine, 4-PO-Psilocin, or 4-PO—HO-DMT), psilocin (4-HO-DMT, 4-hydroxy DMT, psilocine, psilocyn, or psilotsin), baeocystin, norbaeocystin, or salts and isomers thereof; extracts of fungus or other naturally-occurring biological sources of the aforementioned molecules, such as psychoactive or psychedelic mushrooms, including but not limited to species within the biological genera of Panaeolus or Copelandia and Psilocybe (hereinafter referred to as “mushroom-related compounds”);
2. Mescaline (3,4,5-trimethoxyphenethylamine) or salts and isomers thereof (such as mescaline hydrochloride or mescaline fumarate); extracts of peyote (Lophophora williamsii), San Pedro (Echinopsis pachanoi), and Peruvian torch (Echinopsis/Trichocereus peruviana) cactus (hereinafter referred to as “mescaline-related compounds”);
3. N,N-dimethyltryptamine, N,N-DMT, 5-MeO-DMT or salts and isomers thereof (such as DMT fumarate); extracts of plants or other naturally-occurring biological sources of the aforementioned molecules, such as Psychotria viridis, Diplopterys cabrerana, Mimosa tenuiflora (also known as Mimosa hostilis), and Incilius alvarius; extracts traditionally used in ayahuasca brew or tea (hereinafter referred to as “DMT-related compounds”);
4. D-lysergic acid amide (LSA), also-known as Ergine and d-lysergamide or salts and isomers thereof; extracts of plants or seeds of the convolvulaceae or morning glory family (hereinafter referred to as “LSA-related compounds”);
5. Ibogaine; extracts of plants of the Apocynacea family, such as Tabernanthe iboga (or “iboga”) and Voacanga africana (hereinafter referred to as “Ibogaine-related compounds”);
6. Other plant, fungal, or naturally and biologically derived psychedelic compounds; and
7. Combinations thereof.

There are many problems with using plant or fungal compounds as therapeutic treatments or supplements. The shelf life of the product in its raw form is variable and creates a potential for inconsistency in the dose of the active compound in the product. The known concentrations of an active compound in each plant, fungi or batches thereof can vary as well, leading to dosing variability. Taste of consuming the raw form of the plant or fungi is also a major barrier to use. Consumption in raw form can also cause severe digestive upset. Extracting the active compounds and combining them with a solvent can provide a way to control shelf life, product consistency, and amount of active compound to achieve the desired therapeutic effect. Metered-dosing compositions are a way to deliver a known, controlled, and measured dose of the extracted compounds.

Currently there are not many solutions for taking a controlled dose of psychedelic compounds such as the extracts found in ayahuasca brew or tea, psychedelic mushrooms, and peyote. Some of these solutions attempt to instruct users on the amount to take to achieve the desired therapeutic effect, but these solutions fail to meet the needs of the industry because consuming these psychedelic compounds in their raw or natural form are unpredictable and oftentimes dangerous if the consumer is in a situation or state of mind unprepared for the standard dose at psychedelic levels, such as operating a motor vehicle. No current solutions attempt to formulate a consistent and metered dosage. There have been similar metered-dosing solutions related to cannabis, but the extraction and isolation techniques, compositions, and solvents needed are different than for the psychedelic compounds referenced herein due to the polarity of the compounds, vaporization temperatures as related to flash points, and other factors.

Some problems with the application of the psychedelic plant and fungal compounds in the raw state include but are not limited only to the following: 1) the time of onset or “uptake speed” of the therapeutic effects; 2) predictability of dosing; 3) inadvertent stacking of dosing causing effects greater than intended; 4) palatability of taste; 5) shelf life; 6) conditions for crop grows; 7) conditions for extraction; (8) discreetness of possession and use and volume/size of personal possession of multiple doses; (9) ease of accidental ingestion by children; and (10) fear of the unknown or unfamiliar. Additionally, other known forms of processed or extracted compounds, such as vaporizing and inhaling DMT-related compounds or drinking ayahuasca brew or tea, reportedly cause an unwanted or undesirable discomfort in some users.

More specifically, when DMT-related compounds are inhaled after vaporizing with heat, some users complain of the harshness of this technique on the throat and lungs as well as the taste. Some techniques of inhalation of vaporized DMT-related compounds result in molecules closest to the heating element combusting due to variable temperatures of the heating elements, or if a convection heating device is used, combustion can occur by setting the temperature too high above the boiling point of the compound.

Any destruction of the compounds also results in unintended byproducts which can be inhaled which can further irritate the lungs or create unappealing tastes reducing the user repeating the experience or reducing the likelihood of ingesting the intended dose. Also, destruction by heat ultimately reduces bioavailability by virtue of the relevant molecules being destroyed before entering the body. In most inhalation techniques it is difficult or impossible to deliver a predictable dose due to the variability of absorption rates of inhaled vapor, length of inhale, potency or dilution of the substance, etc

Additionally, when one inhales the vaporized compound, a high percentage of the compound is not absorbed into the lungs, resulting in psychedelic compounds being exhaled and therefore bioavailability wasted into the ambient environment.

A metered-dose inhaler (MDI) is a device that delivers a measured amount of an active compound or ingredient as an aerosolized mist the patient can inhale. An MDI generally consists of a pressurized canister of medication in a case with a mouthpiece.

Inhalation of an aerosolized liquid solution containing the DMT-related compounds can be more comfortable and predictable than vaporized compounds. In the liquid state, the aerosolized particles contain multiple molecules of the constituent parts with a greater surface area than inhaled vaporized molecules. The greater mass contained in the aerosolized mist can decrease the likelihood of the lungs providing enough force to cause exhalation of the compounds due to the compounds settling in the lungs. This results in more of the psychedelic compound coming into and staying in contact with lung tissue and thus can increase absorption into lungs rather than remaining in a gaseous state with portions of the intended dose being exhaled. This can increase the bioavailability of the compounds versus a vaporization administration.

This also can have a cost-saving effect by not wasting compounds per dose via exhale and also can allow more of the active compounds to be carried at once. It can also be more convenient for the user to use an MDI device as no heat source is required and less of an exhale smell can be noticed by passersby allowing more privacy for the user. It also requires more hand-eye-coordination for the user than simply puffing on an electronic cigarette or vaporization pen decreasing the likelihood of children accidentally ingesting the compounds.

As with many consumable products that may be moved to and from different environments, a preservative is required for shelf life and stability of the final product. Two common and recommended as safe preservatives in pharmaceutical and natural supplement product formulations are Benzalkonium chloride and Ascorbic acid.

Another impediment to maximizing bioavailability of DMT-related compounds, particularly when traveling through the digestive tract, is the body's endogenous monoamine oxidase (MAO) enzyme which can breakdown DMT through oxidation effectively making it biologically inert before it enters the bloodstream or can potentially reduce the availability of the compound to enter the brain after the psychedelic compound enters the bloodstream. Traditionally, consumers of DMT orally add a monoamine oxidase inhibitor (MAOI) to the DMT-related compound to improve bioavailability through the digestive tract.

Many people who are afraid of mushrooms or other natural sources of these psychedelic compounds, or who would normally be opposed to a “natural product” in general, are more likely to try something that appears in a delivery modality form that they are more familiar with or accustomed to. One such familiar modality is the nasal spray embodiment of the invention. Many individuals who are familiar with nasal sprays are accustomed to an aromatic being included, such as natural or essential oils.

There are currently no known metered dosing modalities with compositions comprising at least one psychedelic compound derived from living organisms, such as plants and fungi, synthetically derived forms or synthetic analogs.

### BRIEF DESCRIPTION

Disclosed herein are new metered-dosing compositions, methods and modalities comprising psychedelic compounds occurring in live organisms, such as plants and fungi. In one embodiment, the composition herein comprises a metered dose of one or more psychoactive and/or psychedelic compounds or synthetic forms or analogues thereof, including but not limited to those derived from total synthesis, function-oriented synthesis, biology-oriented synthesis, complexity to diversity, hybrid molecules, and biosynthesis-inspired synthesis.

### DETAILED DESCRIPTION OF THE PREFERRED EMBODIMENT

Disclosed herein are new compositions and methods comprising a metered-dose(s) of the following “psychedelic compounds” or substances:
1. Psilocybin (O-phosphoryl-4-hydroxy-N,N-dimethyltryptamine, 4-PO-Psilocin, or 4-PO—HO-DMT), psilocin (4-HO-DMT, 4-hydroxy DMT, psilocine, psilocyn, or psilotsin), baeocystin, norbaeocystin, or salts and isomers thereof; extracts of fungus or other naturally-occurring biological sources of the aforementioned molecules, such as psychoactive or psychedelic mushrooms, including but not limited to species within the biological genera of Panaeolus or Copelandia and Psilocybe (hereinafter referred to as “mushroom-related compounds”). Psilocybe cubensis includes Golden Teacher and other common cultivation strains. Psilocybe semilanceata includes liberty caps that are very popular in the wild. Panaeolus cyanescens, aka Copelandia cyanescens is the wild mushroom found in Jamaica. Thirteen species of Panaeolus contain the hallucinogen psilocybin including Panaeolus cyanescens and Panaeolus cinctulus. The bluing hallucinogenic members of this genus are sometimes segregated into a separate genus, Copelandia;
2. Mescaline (3,4,5-trimethoxyphenethylamine) or salts and isomers thereof (such as mescaline hydrochloride or mescaline fumarate); extracts of peyote (Lophophora williamsii), San Pedro (Echinopsis pachanoi), or Peruvian torch (Echinopsis/Trichocereus peruviana) cactus (hereinafter referred to as “mescaline-related compounds”);
3. N,N-dimethyltryptamine, N,N-DMT, 5-MeO-DMT or salts and isomers thereof (such as DMT fumarate, C16H20N2O4); extracts of plants or other naturally-occurring biological sources of the aforementioned molecules, such as Psychotria viridis, Diplopterys cabrerana, Mimosa tenuiflora (also known as Mimosa hostilis), and Incilius alvarius; extracts traditionally used in ayahuasca brew or tea (hereinafter referred to as “DMT-related compounds”);
4. D-lysergic acid amide (LSA), also-known as Ergine and d-lysergamide or salts and isomers thereof; extracts of plants or seeds of the convolvulaceae or morning glory family (hereinafter referred to as “LSA-related compounds”);
5. Ibogaine; extracts of plants of the Apocynacea family, such as Tabernanthe iboga (or “iboga”) and Voacanga africana (hereinafter referred to as “Ibogaine-related compounds”);
6. Other plant, fungal, or naturally and biologically derived psychedelic compounds; and
7. Combinations thereof.

Cannabinoids are expressly excluded from the class of psychedelic compounds listed above and used herein.

As used herein, the term “psychedelic compound,” also known as psychoactive drug or psychotropic substance, refer to a chemical substance that acts primarily upon the central nervous system where it alters brain function, resulting in temporary changes in perception, mood, consciousness and behavior, which includes but is not limited to psychedelic effects. As used herein, the term “psychedelic compound” or “active compound” is meant to comprise one of more of the following: Naturally occurring psychedelic tryptamines, such as O-Acetylpsilocin (4-AcO-DMT), 5-MeO-DMT, bufotenin (5-HO-DMT), psilocybin (4-PO-DMT), and psilocin (4-HO-DMT) or salts and isomers thereof; extracts of plants, fungus or other naturally-occurring biological sources of the aforementioned molecules (hereinafter referred to as “psychedelic tryptamines”); Psilocybin (O-phosphoryl-4-hydroxy-N,N-dimethyltryptamine, 4-PO-Psilocin, or 4-PO—HO-DMT), psilocin (4-HO-DMT, 4-hydroxy DMT, psilocine, psilocyn, or psilotsin), baeocystin, norbaeocystin, or salts and isomers thereof extracts of fungus or other naturally-occurring biological sources of the aforementioned molecules, such as psychoactive or psychedelic mushrooms, including but not limited to species within the biological genera of Panaeolus or Copelandia and Psilocybe (hereinafter referred to as “mushroom-related compounds”). Psilocybe cubensis includes Golden Teacher and other common cultivation strains. Psilocybe semilanceata includes liberty caps that are very popular in the wild. Panaeolus cyanescens, aka Copelandia cyanescens is the wild mushroom found in Jamaica. Thirteen species of Panaeolus contain the hallucinogen psilocybin including Panaeolus cyanescens and Panaeolus cinctulus. The bluing hallucinogenic members of this genus are sometimes segregated into a separate genus, Copelandia; Mescaline (3,4,5-trimethoxyphenethylamine) or salts and isomers thereof (such as mescaline hydrochloride or mescaline fumarate); extracts of peyote (Lophophora williamsii), San Pedro (Echinopsis pachanoi), or Peruvian torch (Echinopsis/Trichocereus peruviana) cactus (hereinafter referred to as “mescaline-related compounds”); N,N-dimethyltryptamine, N,N-DMT, 5-MeO-DMT or salts and isomers thereof (such as DMT fumarate, C16H20N2O4); extracts of plants or other naturally-occurring biological sources of the aforementioned molecules, such as Psychotria viridis, Diplopterys cabrerana, Mimosa tenuiflora (also known as Mimosa hostilis), and Incilius alvarius; extracts traditionally used in ayahuasca brew or tea (hereinafter referred to as “DMT-related compounds”); D-lysergic acid amide (LSA), also-known as Ergine and d-lysergamide or salts and isomers thereof; extracts of plants or seeds of the convolvulaceae or morning glory family (hereinafter referred to as “LSA-related compounds”); Ibogaine; extracts of plants of the Apocynacea family, such as Tabernanthe iboga (or “iboga”) and Voacanga africana (hereinafter referred to as “Ibogaine-related compounds”); Other plant, fungal, or naturally and biologically derived psychedelic compounds; and; combinations thereof.

As used herein, the term “processing” refers to the treatment or preparation of a raw or organic material by a special action or method that produces a change or development. As used herein, the term “extracting” or “extraction” refers to the various methods used to get, pull, or draw out a specific compound from the raw form or composition. As used herein the term “processing” or “extraction” also refers to the removal of active components from inactive components. As used herein, the term “active components,” includes the “psychedelic compounds” referenced above. As used herein, the term “inactive components” refers but is not limited to proteins, lipids, sugars, cellulose, and chitin, which can be found in organic matter.

As used herein, “synthetic,” “synthetically,” or “synthetic forms,” refers to a compound formed through a chemical process by human agency, as opposed to those of natural origin, which includes but is not limited to psychedelic or psychoactive compounds derived from chemical syntheses (chemical reactions outside of the natural biological processes) or compounds created from chemical reactions in other genetically-modified organisms, such as bacteria, algae, or yeast fermentation (aka “synthetic biology methods” or “biologically derived”).

As used herein, the term “amount” or “particular amount” refers to the quantity of a compound or compounds. In one embodiment, an amount is the combined quantity of two compounds within a sample. In one embodiment, an amount is measured by weight. In one embodiment, an amount is measured by dry weight.

As used herein, the term “ratio” refers to the amount of a compound in relation to the amount of another compound or compounds. In one embodiment, there is a 1000:1 to 1:1000 weight to weight ratio of a psilocybin derivative to solute.

As used herein, the terms “formulation”, “composition” or “feedstock” refers to a resulting product from combining one or more ingredients or elements.

As used herein, the term “dry weight” refers to a measurement of the mass of a sample after removing all, or substantially all, the liquid from the sample. In one embodiment, removing liquid comprises dehydrating, heating, stirring, filtering, and/or any other method suitable for liquid water. In one embodiment, dry weight is measured by pounds. In one embodiment, dry weight is measured by ounces. In one embodiment, dry weight is measured by grams, e.g., micrograms, milligrams, kilograms, etc. In another embodiment, a solvent or liquid that is not water such as ethanol is deemed to have less than a significant amount of water present.

As used herein, the term “dry weight basis” refers to the measure of a dry weight of a sample as defined above divided by the total mass of the composition, solution or formulation. This is frequently but not exclusively expressed as a percentage or ratio.

As used herein, the term “nasal inhalation” refers to drawing in by breathing through the nasal passage. As used herein, the term “nasal inhalation” can refer to breathing or taking in through the nasal passages with or without the assistance of a pump or pressurized gas. As used herein, nasal inhalation or nasal application of a composition via the aerosol or spray generated by the nasal spray device with or without the user inhaling, although the term nasal inhalation is utilized for any variation of the nasal route of administration,

As used herein, the term “oral inhalation” refers to drawing in by breathing through the mouth. As used herein, the term “oral inhalation” refers to breathing through the oral passage with or without the assistance of a device which generates an aerosol with a mass median diameter between 100 nm and 7 mcm measured in accordance the United States Pharmacopiea that deposits active compounds anywhere in the respiratory tract between the mouth and the deep lung.

As used herein, the term “mucosal absorption” refers to the taking in of a substance through the mucosal membranes. As used herein, the term “transmucosal” also refers to the taking in of a substance through the mucosal membranes.

As used herein, the term “oral ingestion” refers to the act of taking in through the mouth for digestion.

As used herein, the term “topical” refers to the application of something to a particular or local part of the body, particularly but not limited to the skin. Furthermore, the term “transdermal” is also meant to encompass topical application. As used herein, “topically applied” or “topically administered” or “transdermal” refers to providing a product which compounds active ingredients in a manner which enters the blood or body through the dermis, or skin. In one embodiment, “transdermally administered” or “transdermal application” comprises applying a composition to the skin, e.g., applying a topical composition, applying a liquid or emulsion composition, etc. In one embodiment, transdermally administering a composition comprises applying a patch embedded with the composition or formulation to the skin.

As used herein, the term “tablet” or “capsule” refers to a small piece of various shape or a gelatinous case enclosing a dose of medicine to be taken orally.

As used herein, the term “monoamine oxidase inhibitors” also known as MAOI refers to various substances that block the enzymatic breakdown of certain monoamine neurotransmitters. As used herein, the term “MAOI” refers to a molecule binding to a monoamine oxidase enzyme thereby reducing the activity of the enzyme.

In one embodiment, the formulations disclosed herein comprise a monoamine oxidase inhibitor. In certain embodiments of the invention containing DMT-related compounds, an MAOI is added to the formulation to increase bioavailability if microdosing throughout the day. The preferred MAOI compound is harmaline or other harmala alkaloids.

As used herein, the term “sublingual” refers to an application of a composition or medicine to the area underneath the tongue of an organism.

As used herein, the term “water soluble” refers to a compound capable of dissolving in water at standard temperature and pressure. In one embodiment, a compound's solubility in water is an inherent property of a compound. In one embodiment, a compound's solubility in water is facilitated by another compound, e.g., an excipient.

As used herein, the term “solvent” refers to a compound or composition that dissolves another (solute), thereby creating a solution. In one embodiment, the solvent is water. In one embodiment, the solvent is alcohol. As used herein, the term “aqueous solution” refers to a solution in which the solvent is water with a compound dissolved or uniformly dispersed.

As used herein, the term “homogeneous” refers to a solid, liquid, or gaseous composition that has two or more compounds present within one state or thing, e.g., a clear colorless solution. In one embodiment, the homogenous mixture disclosed herein has the same proportion, concentration, and/or ratio of its components across different samples. In one embodiment, the components in the homogenous mixture are in the same state of matter. In one embodiment, a homogenous mixture comprises one or more compounds within a solution, e.g., psilocybin and a solvent.

As used herein, the term “dose” or “dosing” refers to a quantity of medicine desired to be taken at one time or to administer or to take a dose of medicine.

As used herein, the term “metered dosing” or “metered dose” refers to a delivery of a specific amount of the desired compounds per application via various delivery forms including but not limited to inhalation, oral ingestion, nasal spray, mucosal membrane absorption, transmucosal, buccal or sublingual application, or transdermal application.

As used herein, the term “nasal spray device” refers to a device comprising a container, vial, or reservoir; an actuator, liquid transfer pump or other means of squeezing or forcing fluid out of a reservoir, vial, or container; an orifice; a channel or tube fluidly connecting the canister to the orifice such that a fluid emitted from the reservoir, vial, or container is channeled through the tube and out of the orifice and applied to the nasal passage(s).

As used herein, the term “liquefied gas” refers to a substance which at standard temperature (0 degrees centigrade) and pressure (1 bar) exists as a gas phase, but it either compressed or cooled to exist as a liquid phase, such as 1,1,1,2-Tetrafluoroethane (HFA 134a) and 1,1,1,2,3,3,3-Heptafluoropropane (HFA 227) and butane.

In one embodiment, transmucosal application comprises the new composition being applied to and entering through, or across, a mucous membrane, e.g., an oral administration of a composition to the throat, gums or tongue. In one embodiment, transmucosally applying refers to delivering a drug via the cavity between the cheek and gum, e.g., a liquid composition, a fast dissolving tablet or thin film.

As used herein, the term “psilocin” refers to the psychedelic compound found in certain mushroom species or synthesized from compounds found in certain mushroom species, which is known to have psychedelic properties. Psilocin has a chemical formula: C₁₂H₁₆N₂O and structure:

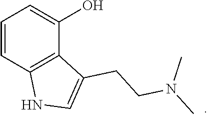

As used herein, the term “psilocybin” refers to the compound found in certain mushroom species, it is also the phosphorylated embodiment of psilocin. Psilocybin has a chemical formula: C₁₂H₁₇N₂O₄P and structure

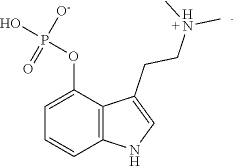

As used herein, the term “baeocystin” refers to the compound commonly found in certain mushroom species. Baeocystin is a chemical analog of psilocybin with a chemical formula: C₁₁H₁₅N₂O₄P and structure:

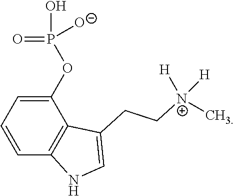 [SMILES: C[NH2+]CCc1c[nH]c2cccc(OP(=O)([O-])O)c12]

As used herein, the term “norbaeocystin” refers to the compound commonly found in certain mushroom species. Norbaeocystin is a chemical analog of psilocybin with a chemical formula: C₁₀H₁₃N₂O₄P and structure:

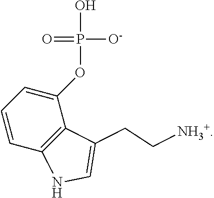

As used herein, the terms “mescaline” and “3,4,5-trimethoxyphenethylamine” are interchangeable and refer to the compound found in certain species of cacti. Mescaline has a chemical formula: C₁₁H₁₇N O₃ and structure:

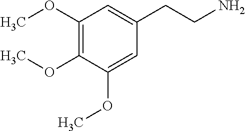

As used herein, the terms “DMT”, “N,N-dimethyltryptamine”, and “N,N-DMT” are interchangeable and refer to the compound found in certain species of plants and animals and can have a psychedelic effect. DMT is an analog of many psychedelic compounds and occurs within serotonin and melatonin. DMT has a chemical structure of C₁₂H₁₆N₂ and structure:

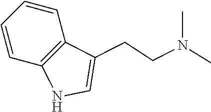

As used herein, the terms 5-MeO-DMT and 5-methoxy-N,N-dimethyltryptamine are interchangeable and refer to the compound found in certain species of plants and animals and can have a psychedelic effect. 5-MeO-DMT is a structural analog of many psychedelic compounds naturally occurring for important biological functions such as serotonin and melatonin. 5-MeO-DMT has a chemical structure of C₁₃H₁₈N₂O and structure:

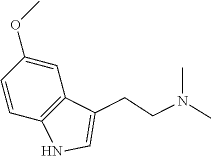 [SMILES: COc1ccc2[nH]cc(CCN(C)C)c2c1]

As used herein, the terms “Ergine”, “LSA”, and “d-lysergic acid amide” are interchangeable and refer to the compound commonly found in certain species of vine plants and can have a psychedelic effect. Ergine has a chemical formula: C₁₆H₁₇N₃O and structure:

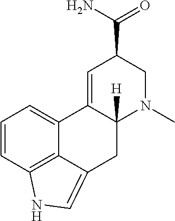

As used herein, the term “ibogaine” refers to the compound found in some species of plants, it is known for its psychedelic properties. Ibogaine has a chemical structure: C₂₀H₂₆N₂O and structure:

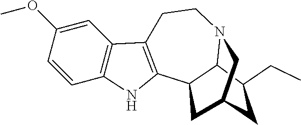

Metered dose compositions have been shown to achieve significant therapeutic effects, while limiting negative side effects associated with the intake of too much or too little of the intended active compound.

The new compositions and methods of use provide a controlled dose of an extracted, and more purified psychedelic compound than what exists in their respective natural raw biomass or dried or dehydrated form of the raw biomass. The compositions more specifically include a type of psychedelic compound as defined above.

Extracting the active compound from the raw plant or fungal form and formulating it to be administered in a controlled and/or metered dosing form can eliminate the dosage consistency, batch-to-batch variability, and other problems associated with raw form or other types of consumption. Metered dosing is a way for the subject to receive a controlled dose of a compound. Nasally and orally inhaled, ingestible, nasal sprays, mucosally absorbed, sublingual, and topically administered forms of supplements and other compounds have shown consistent and acute effects.

There is a greater new user rate observed with the convenience of metered dosing solutions as it removes the fear of inconsistent dosing and adds the familiar routes of administration resulting in more user compliance with treatment rather than ingesting raw biomass, the traditional method.

With the faster uptake speeds of certain embodiments and increased ease of dosing, it is less likely that users will accidentally overdose or otherwise consume more than they intended. In some cases, when someone consumes more than desired there may be an unintended psychological or physiological effect that puts the user and the public at risk due to accidently reaching impairment levels and then driving or operating heavy machinery or in regards to other important activities when someone needs to be at their peak performance (i.e., parenting, employment, etc.).

With metered-dosing solutions often having more complex biomechanical actions to administer (i.e., greater hand-eye-coordination or dexterity than simply placing biomass in one's mouth), metered dosing is potentially less likely to be accidently ingested or misused by children or adults, which can often be the case for eating biomass or when obscuring ground biomass in a food product, such as a candy that can be easily mistaken as a benign food item.

Processing the raw form to extract and further isolate extracted compounds and formulating the active compounds into compositions to be contained within specific metered-dosing delivery modalities with consistent concentrations of active compounds per dose can be administered nasally, orally, transmucosally, buccally, sublingually, topically or by inhalation. This mitigates adverse or unintended experiences from uncontrolled dosing.

Some limited and specific examples of the methods of use for particular conditions or therapeutic effect are: topical psychedelic compound extracts applied behind the ear could help with motion sickness; nasal and inhaled preparations could help with psychotherapy analysis with a small, fast-acting dose for the psychotherapy session; sublingual preparations may have more of a muscle relaxing effect than orally administered versions and precisely manufactured oral dosage forms such as capsules, gel cap or tablets. At the cellular level, the 5-HT2A receptor which mediates psilocybin's psychedelic effects, also mediates several important cardiovascular effects in both the peripheral nervous system and central nervous system, including vascular smooth muscle contraction, platelet aggregation, thrombus formation and coronary artery spasm. The selective 5-HT2A antagonists ketanserin, AT-1015, and sarpogrelate all have vasodilating properties. (Kyriakides et. al., 1999; Kihara et al., 2000; Miyata et al. 2000). Methysergide, a 5-HT2A/2C receptor antagonist (and migraine prophylactic agent), inhibits the vasoconstrictive and pressor effects of serotonin as well as the serotonin actions on extravascular smooth muscle. Conversely, the 5-HT2A agonist DOI constricts rat veins, an effect that is blocked by the 5-HT2A antagonist ketanserin (Cohen et al., 1993).

All preparations and delivery preparations included herein may have improved compliance due to lack of taste in cases where swallowing the raw form has been known to be unpleasant, unpredictable and a major deterrent to use.

Embodiments administered nasally in particular have several advantages over traditional ingestion methods. “When medications of appropriate molecular character and concentration are delivered intranasally, they are quickly transported across this capillary network and delivered to the systemic circulation, thereby avoiding the absorption-limiting effects of first-pass metabolism.” Corrigan M, Wilson S S, Hampton J. Safety and efficacy of intranasally administered medications in the emergency department and prehospital settings. Am J Health Syst Pharm. 2015; 72(18): 1544-1554. doi:10.2146/ajhp140630. “Active pharmaceutical ingredients absorbed in the nasal mucosa can not only have local and/or systemic effect, but they can also be used to target the brain directly (Gartziandia et al., 2016; Singh et al., 2016). The nasal route of drug administration has several advantages over oral or intravenous administration, which include non-invasiveness, self-administration, shorter time to onset of effect and higher bioavailability due to avoidance of hepatic first-pass metabolism. Moreover, bypassing the [blood-brain barrier] may potentially increase central nervous system (CNS) availability of the drug (Dufes et al., 2003).” Erdö F, Bors L A, Farkas D, Bajza Á, Gizurarson S. Evaluation of intranasal delivery route of drug administration for brain targeting. Brain Res Bull. 2018; 143:155-170. doi:10.1016/j.brainresbull.2018.10.009.

The present invention is directed to metered dosing compositions of biologically occurring psychedelic plant and fungal compounds for oral and nasal inhalation; transmucosal, buccal, and sublingual; and topical applications.

The invention relates generally to a metered dosing composition and method of use. Metered dosing is a way to deliver a known and measured dose.

Provided herein are formulations and delivery modalities for metered-dosing that provide for a more comfortable and predictable subject experience.

In its most basic form, the composition is made up of the following components a) extracted psychedelic compound; and b) solvent. These components are related as follows: the combination of the psychedelic compound and the solvent allow for the metered dosage by a specific method of delivery. It should further be noted that the solvent used may vary according to each method of delivery. It should be noted that the methods for extracting the psychedelic compounds from the raw form or biomass may vary. The combination of the psychedelic compound and the solvent will be delivered via various methods that will be specifically dosed for the desired treatment or effect. Ease of dosage adjustment is an additional component with the use of psychedelic compounds as a treatment.

It will nevertheless be understood that no limitation of the scope of the invention is thereby intended. Any alterations and further modifications of the inventive feature(s) described herein, and any additional applications of the principals of the invention as described herein, which would normally occur to one skilled in the relevant art and having possession of this disclosure, are to be considered within the scope of the invention claimed.

The forms of performing the method of delivery associated with the disclosed compositions comprise the following: oral inhalation; nasal inhalation; oral ingestion; nasal spray; transmucosal, buccal, or sublingual application/delivery; or topical application/delivery. It should further be noted that each method of delivery/application may require a specific composition of dosage, amount of psychedelic compound, specific type of solvent respectively, as well as other added compounds.

Different features, variations and multiple different embodiments are included herein and described with various details. What has been described in this application at times in terms of specific embodiments is done for illustrative and descriptive purposes and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by this disclosure. It is indeed intended that the scope of this disclosure should be determined by a proper legal interpretation and construction of the disclosure, including equivalents, as understood by those of skill in the art relying upon the complete disclosure present at the time of filing.

The embodiments of the present invention contemplate dosage forms with a total weight of between approximately 10 mcg to 500 mg of psychedelic mushroom-related compound(s) and extracts, depending on the composition of the active compound(s) and method of delivery. This dosage form can be used for all natural, salt or isomers, semi-synthetic and synthetic derivatives, and analogues of the aforementioned fungi or mushrooms and their mushroom-related compounds. Handling and processing of the extract is significant in the proper delivery of the active compounds with the associated solvents and additives, which synergistically work to improve the medicinal or therapeutic value of the plant or fungi chosen for the particular ailment or condition under treatment.

The embodiments of the present invention contemplate dosage forms with a total weight of between approximately 10 mcg to 500 mg of DMT-related compounds, DMT or 5-MeO-DMT, depending on the composition of the active compound(s) and method of delivery. This dosage form can be used for all natural, salt or isomers, semi-synthetic and synthetic derivatives, and analogues of the aforementioned DMT-related compounds. Handling and processing of the extract is significant in the proper delivery of the active compounds with the associated solvents and additives, which synergistically work to improve the medicinal or therapeutic value of the plant or fungi chosen for the particular ailment or condition under treatment.

The embodiments of the present invention contemplate dosage forms with a total weight of between 50 mcg to 900 mg of psychedelic mescaline-related compounds or 70 mcg to 1100 mg of mescaline, depending on the composition of the active compound(s) and method of delivery. This dosage form can be used for all natural, salts or isomers, semi-synthetic, synthetic derivatives, or analogues of the aforementioned mescaline-related compound. Handling and processing of the extract is significant in the proper delivery of the active compounds with the associated solvents and additives, which synergistically work to improve the medicinal or therapeutic value of the plant or fungi chosen for the particular ailment or condition under treatment.

The embodiments of the present invention contemplate dosage forms with a total weight of between 0.04 mg to 700 mg of psychedelic LSA-related compounds. This dosage form can be used for all natural, salt or isomers, semi-synthetic and synthetic derivatives, and analogues of the aforementioned LSA-related compound.

An embodiment of the present invention is a composition of a psychedelic compound or combination of compounds in an aqueous solution that is delivered via a nasal spray device. The nasal spray device includes a vial, vessel or container serving as a reservoir for the composition or formulation, an actuator or pump-type portion, a channel or tube portion with an exit office to apply the composition to the nasal passages intended for the composition or formulation to enter the nasal and/or sinus passageways via the force generated by the nasal spray device with or without the user inhaling, although the term nasal inhalation is utilized for any variation of the nasal route of administration.

In some embodiments, the metered-dose nasal spray formulation is provided. The formulation can comprise at least one psychedelic compound in an aqueous solution, to be administered via a nasal spray or nebulizer. The formulation can further comprise a thickening agent. The formulation can further comprise a preservative.

In some embodiments of the invention, the patient is instructed to administer a dose from a nasal inhaler to each nostril the first time that the invention is utilized. In some embodiments containing psilocybin, the patient is then instructed to wait at least twenty minutes before re-administration to prevent consuming hyper-therapeutic amounts of the active compounds. In some embodiments, containing psychedelic compounds, the patient is instructed to wait at least five minutes before re-administration to prevent consuming hyper-therapeutic amounts of the active compounds. Once a patient self-titrates on to the desired effect and is self-aware of the level of preferred efficacy or perceptible effects or lack of effects, the protocol for self-administration can be reduced to between two and five minutes between sprays for each nostril to allow for maximum absorption.

In some embodiments of the nasal spray, an aromatic terpene, such as linalool, pinene, fencholl, Geraniol, monoterpenes, diterpenes, sesquiterpenes and mixtures thereof is added to the formulation along with the psychedelic compound. In some embodiments there are less than 10 parts terpene to one part active ingredient. In some embodiments there are less than 1 part terpene to one part active ingredient. In some embodiments there are less than 0.1 part terpene to one part active ingredient. In some embodiments there are less than 0.03 parts terpene to one part active ingredient. In some embodiments there is less than 0.004 part terpene to one part active ingredient. In some embodiments where the extracted compounds also include other natural or inactive mushroom compounds an aromatic can cover up or disguise the smell of mushrooms.

In some embodiments of the nasal spray invention, a preservative, such as Benzalkonium chloride, ascorbic acid, or any other biocompatible excipient that increases product shelf life thereof is added to the formulation along with the psychedelic compound. In some embodiments, the ratio of preservative ingredient is 10:1 to the active ingredient. In some embodiments, the ratio of preservative ingredient is 1.3:1 to the active ingredient. In some embodiments, the ratio of the preservative ingredient is 0.02:1 to the active ingredient.

Some embodiments of the invention include incorporating into the formulation a surfactant. In some of the embodiments, the surfactant included in the formulation is an emulsifying agent containing a combination of hydrophilic and hydrophobic substituents, particularly a combination of fatty acid esters of polyethylene glycol, ethoxylated glycerol and polyethylene glycol, glycerol polyethylene glycol ricinoleate, phospholipids, and mixtures thereof.

Some embodiments of the invention include a polymer. In some embodiments the polymer is a thickening agent to decrease the likelihood that the solution in the nasal spray modality runs out of the nostril or down the throat, utilizing such compounds as a hydroxypropyl cellulose, hydroxymethyl cellulose, polyethylene glycol, or other water soluble polymers. In other embodiments, a polymer enables transdermal delivery the active ingredient. In one embodiment, naturally-occurring lipids and or proteins are left in the extract for the purpose of thickening. In some embodiments, the thickening agent is 20% w/w of thickening agent to the weight of formula. In some embodiments, the thickening agent is 10% w/w of thickening agent to the weight of formula. In some embodiments, the thickening agent is 5% w/w of thickening agent to the weight of formula. In some embodiments, the thickening agent is 1% w/w of thickening agent to the weight of formula. In some embodiments, the thickening agent is 0.5% w/w of thickening agent to the weight of formula. In some embodiments, the thickening agent is 0.1% w/w of thickening agent to the weight of formula.

In accordance with the various embodiments of the invention, a formulation can comprise of a psychedelic compound and water in combination with a preservative, aromatic terpene, emulsifying agent, thickening agent which are combined in relative quantities to result in a stable solution.

In a preferred embodiment of the mushroom-related nasal spray, the formulation can be configured such that the mushroom-related compounds forming the active ingredient make up 10% to 0.1% by weight of the formulation.

In one embodiment, an inactive mushroom ingredient, such as biocompatible lipids and proteins including but not limited to phospholipids or cholesterol, is included. In this example, the phospholipids serve as encapsulating agents and the proteins serve as surfactants. In this embodiment, the inactive mushroom ingredients are present in a ratio of inactive to active of 1:1.

In some embodiments, mushroom extract is dissolved in water to 0.5% weight by weight concentration of extract to 20% w/w concentration. In some embodiments, the psychedelic-compound formulation is transferred to a vial equipped to discharge a nasal dose between 50 mcl and 300 mcl.

An embodiment of the present invention is a new composition that is delivered via nasal inhalation. An embodiment of the present invention is a new composition that is delivered via a topical form. An embodiment of the present invention is a new composition that is administered via transdermal application. An embodiment of the present invention is a new composition that is delivered in a tablet or capsule form. An embodiment of the present invention is a new composition that is delivered by oral inhalation. An embodiment of the present invention is a new composition that is administered by transmucosal application.

Provided herein are formulations of psychedelic compounds for transmucosal, buccal, or sublingual delivery. A composition or formulation is provided in some embodiments. In some embodiments, the psychedelic compound is present at a concentration of 0.001% to 75% by weight of the composition and/or formulation. In some embodiments, a combination of one or more psychedelic compounds are present at a total concentration of 0.001% to 90% by weight of the composition and/or formulation. An embodiment of the present invention is a new composition that is delivered via absorption through the mucosal membranes.

In some embodiments, the formulations or compositions are provided in a suitable delivery modality or formulation for administering dosing to a subject, such as but not limited to a sublingual film, tablet, capsule, lozenge, troche, tincture, transdermal patch, topical lotion, dry powder inhaler, thermal vaporizer (electronic cigarette), air jet nebulizer, vibrating mesh nebulizer, vaporizer, or pressurized metered dose inhaler. In some embodiments, the psychedelic compound is present at 3 mcg to 800 mg per individual formulation or dose. In some embodiments, the dose of psychedelic compounds is 7 mcg to 600 mg in each individual product that is capable of being consumed by or administered to a subject. In some embodiments, the total dose of psychedelic compounds is 10 mcg to 1200 mg

In some embodiments, a “sublingual film” or a thin film refers to a substrate containing the psychedelic compounds and hydrophilic material as a solid solution or suspension and optionally constructed as a multilayer film that dissolves in the mouth for transmucosal, buccal or sublingual administration of doses that has dissolves in 100 ml of distilled water at 37 degrees centigrade within 30 minutes while testing for dissolution in accordance the United States Pharmacopoeia, and is greater than 20 mcm in thickness, but less than 2 cm in thickness with a surface area between 1 and 12 cm2, In some embodiments the film is prepared and cut into single-dose strips with psychedelic compounds on a hydrophilic polymers and optionally a combination of one or more of a surfactant, such as benzalkonium chloride, benzethonium chloride, sodium lauryl sulphate, and a sweetener.

In some embodiments, the formulation is comprised of any one or more of a preservative, a buffer, a chloride salt, a biocompatible polymer, a carbohydrate, a solvent, a terpene, a surfactant, a liquified gas, a solvent, and combinations thereof.

An embodiment of the present invention is a new composition that is delivered via an oral inhaler consisting of a pressurized metered dose inhaler (pMDI), dry powder inhaler (DPI), vibrating mesh liquid nebulizer, air jet liquid nebulizer, thermal inhalers such as electronic cigarettes or any device that produces an aerosol with a mass median aerodynamic diameter between 100 nm and 7 mcm as tested in accordance with United States Pharmacopeia and is less than 2 kg in total weight

An embodiment of the pMDI is comprised of a psychedelic compound, a sufficient amount of a polar solvent such as ethanol to dissolve the active compound, and an amount of propellent of at least one of 1,1,1,2-Tetrafluoroethane (HFA 134a) and 1,1,1,2,3,3,3-Heptafluoropropane (HFA 227). In various embodiments a variety of propellants can comprise the formulation. In some embodiments of the pMDI, the formulation is dispensed into any canister. In some embodiments of the pMDI the canister provided is an aluminum aerosol canister. In some embodiments of the pMDI, the formulation provided herein comprises an orifice and actuator configured to distribute aerosol particles with a diameter between 0.25 and 6 micron.

In a preferred embodiment of the DMT-related compound pMDI, the formulation and device delivery modality provide a dose per aerosolized actuation containing 1 mg of at least one of DMT freebase, fumarate, and combinations thereof.

It is an object of the present invention to provide a composition comprising a psychedelic compound for nasal spray application.

It is an object of the present invention to provide a composition comprising a psychedelic compound for oral ingestion.

It is an object of the present invention to provide a composition comprising a psychedelic compound for mucosal membrane, buccal, or sublingual absorption.

It is an object of the present invention to provide a composition comprising a psychedelic for transdermal application.

It is an object of the present invention to provide a composition comprising a psychedelic compound for oral inhalation.

It is an object of the present invention to provide access to a controlled and known dose of a psychedelic compound.

It is an object of the present invention to provide access to a controlled and known dose of a combination of psychedelic compounds via a single delivery modality.

It is an object of the present invention to provide access to a controlled and known dose of a combination of psychedelic compounds via multiple delivery modalities.

It is an object of the present invention to mitigate stomach and digestive upset from consuming raw plants, mushrooms, or other biomass.

It is an object of the present invention to mitigate temporary or permanent psychosis or extreme and unintended intense psychedelic effects or unintended altered states of consciousness caused by too large of a dose of psychedelic compound than intended.

It is an object of the present invention to improve user's mental health.

It is an object of the present invention to improve user's physical health.

It is an object of the present invention to improve user's mental focus.

It is an object of the present invention to mitigate feelings of anxiety.

It is an object of the present invention to facilitate self-reflection and introspection without unintended physiological responses that produce increased adrenaline or cortisol responses.

Although the invention has been described in detail with reference to several embodiments, additional variations and modifications exist within the scope of the spirit of the invention as described.

Although the disclosed invention has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Where reference is made to a particular compound, it should be understood that this disclosure also contemplates salts and derivatives of that compound as well as degradation products, such as oxidized versions of explicitly disclosed molecules.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

EXAMPLE 1. Manufacturing of Mushroom-Related Compound Nasal Spray. In some embodiments, Psilocybin-containing mushroom fruiting bodies were removed from the live organism by cutting the stem of the fruiting bodies near the mycelium-containing substrate. The resulting live biomass was sliced into sections and uniformly spread out over a porous metal grate. Heated ambient air was blown over the mushroom biomass over time to dry the biomass. The resulting biomass was ground and pulverized.

The biomass was then boiled in 200-proof USP-grade ethanol for at least 20 minutes while agitating the feedstock in an ultrasonic bath. The ratio of dry weight ground mushroom biomass to ethanol was at a ratio of 1:3 weight by weight ratio. The extracted compounds were dissolved into the ethanol and the remaining undissolved biomass was removed via a two step filtration process. The first step employed a coarse mesh and gravity filtration. The second step employed vacuum filtration with the aid of buchner funnel equipped with a 0.2 micron filter paper. The filtrate was mixed with water at a ratio of 2 parts filtrate to 1 part distilled water by volume and heated to a maximum of 78 degrees centigrade with agitation for at least 20 minutes.

The resulting feedstock was centrifuged at 1400 Relative Centrifugal Force (RCF) for 10 minutes. This supernate was a clear liquid and the pellet was dark brown. Using a syringe the clear supernate containing the psychedelic fraction was removed. Thereafter the clear supernate was evaporated in a heated ultrasonic bath at 50 centigrade and ambient pressure.

The extract was dissolved in water to 1% weight by weight concentration of extract. The solution was transferred to a vial equipped with a 125 mcl per dose nasal spray. Nominally, this delivered 1.3 mg of extract per actuation of the nasal spray.

In one embodiment of the invention, 11 human subjects self-administered 1.3 mg of psilocybin-containing mushroom extract into each nostril.

All subjects were questioned in intervals up to three hours after use and no adverse events were reported, including no fainting, disorientation, or noticeable or perceptible signs of adverse effect of the mental or physical condition. Subjects were lucid and sentient and displayed normal cognitive abilities. Furthermore, there were no reported symptoms of upset stomach, nasal burning, or unintended psychedelic effects.

Subject A ingested six total sprays, two at a time at fifteen-minute intervals, and after thirty minutes reported a “slight body hum.” Subject A continued the experiment at the same ingestion interval and after one hour and fourteen sprays. Subject A reported improved “focus” and feeling “less anxious.” After twenty-four sprays over two hours and fifteen minutes, Subject A reported feeling a significantly heightened mood, a continued “body buzz,” continued feelings of focus, and feeling “more creative.”

Subject B, following the same time and dosage interval of Subject A, reported “improved mood” after 45 minutes. After ingesting a total of eighteen sprays over two hours and fifteen minutes, Subject B reported improved focus and creativity lasting approximately two hours past final application.

Subject C who self-reported as a “frequent” and “recent” raw biomass microdoser reported after 45 minutes of dosing feeling “improved focus” but credited that not to the dosing but equated it to past similar results when similarly situated in a controlled setting without distractions while practicing mindfulness for 45 minutes, which was what the study was akin to.

Subject D ingested one spray per nostril at the beginning of the experiment. Subject D reported a perceptible feeling throughout the body at the twenty-minute mark that lasted for approximately two hours. Subject D self-reported an increased feeling of “well-being” and a decrease in stress and feelings of anxiety

Subject E administered two sprays per nostril in immediate sequence at the beginning of the experiment. Onset was reported after fifteen minutes post-application. Subject E reported feelings of well-being, decreased anxiety, improved mood, focus and relaxation.

Subjects F and G each took a single spray to the nostril and reported no perceptual effects positive or negative.

Subject H reported a “mellow” and “calming” feeling after fifteen minutes lasting for forty-five minutes.

Subject I reported it was “pleasant” with no complaints.

Subject J, a self-described experienced user of psilocybin at microdosing and psychedelic dosing levels without recent use, reported perceptible effects after thirteen minutes following one spray in each nostril. Subject J noted an “enjoyable aura” of the “microdosing experience.”

Subject K, another self-described “experienced microdoser” reported mild effects three minutes after ingestion from a single spray to each nostril. Fifteen minutes after ingestion the subject reported a feeling of “total body relaxation” and recanted the reported perception of “mild effects” after three minutes believing he was mistaken due to being hyper focused on what his body was feeling in the moment. Comparing the three minute and fifteen minute report, the subject reported that the latter report was clearly a very pleasant effect.

EXAMPLE 2— DMT NASAL SPRAY. A 1:1 slurry by dry weight of finely ground dried mimosa hostilis root and 5% by volume acetic acid in water is mixed. An amount of boiling distilled water equal to the amount of dilute acetic acid solution is added slowly over 5 minutes with a resulting acetic acid concentration of 2.5% by volume. This feedstock is mixed manually every 5 minutes over the course of 45 minutes. An amount of calcium hydroxide equal to 75% by weight of the mass of mimosa hostilis root is added to the feedstock and the red color of the feedstock has dissipated. The feedstock is transferred into a suitable vessel in a 90 degree centigrade water bath. An amount of naphtha equal to the weight of mimosa hostilis root is added and this feedstock is mixed manually every minute over 10 minutes forming a paste-like consistency. After 10 minutes, the naphtha is decanted into a flat bottom dish. This naphtha extraction is repeated an additional 2 times combining decanted fractions. The decanted fractions are chilled to −5 centigrade for 12 hours. After this crystallization period, the naphtha is decanted and precipitate of isolated DMT freebase is recovered and dried at ambient conditions to remove residual solvent. A 20 mg per ml solution of DMT in acetone is prepared. An amount of fumaric acid is added to achieve 6.2 mg per ml concentration. The solution is briefly mixed and allowed to fully evaporate at ambient conditions to yield isolated DMT fumarate.

The DMT fumarate is dissolved in water to 5% weight by weight concentration. The solution is transferred to a vial equipped with a 125 mcl per dose nasal spray. This will deliver 6.5 mg of active compound per actuation of the nasal spray.

EXAMPLE 3— MESCALINE NASAL SPRAY. San Pedro cactus (Echinopsis pachanoi) is dried and ground. A slurry in the ratio of 4:12:1 dry weight of cactus to distilled water to dry weight calcium hydroxide is prepared and well mixed for 7 minutes. A volume of limonene equal to the amount of distilled water is added to the feedstock. For 3 hours the slurry is mixed every 15 minutes in ambient conditions. The limonene fraction is decanted after 3 hours and vacuum filtered through a buchner funnel equipped with 0.2 mcm filter paper. To the filtrate an equal mass of 5% by volume acetic acid in water is mixed relative to the amount of calcium hydroxide employed. Mix thoroughly for 5 minutes and transfer to a separatory funnel. Collect the aqueous bottom fraction which contains mescaline acetate. Evaporate in a convection oven at 70 degrees centigrade.

The mescaline acetate is dissolved in water to 7% weight by weight concentration. The solution is transferred to a vial equipped with a 125 mcl nasal spray. This will deliver 8.8 mg of active compound per actuation of the nasal spray.

EXAMPLE 4— DMT ORAL INHALER. A feedstock is prepared comprising of 180 mg of DMT freebase per ml in ethanol. 1.2 g of feedstock is dispensed into a 20 ml aluminum aerosol canister from Presspart. The canister is crimped with a 50 mcl BK357 valve from Bespak utilizing a Pamasol Small Scale Production Plant 2005/021. Canisters are filled with 12.8 g of HFA 134a via overpressure through the valve stem. The formulated canister is then inserted into a BK634 actuator with a 0.3 mm orifice diameter. This product aerosolizes 1 mg of DMT freebase per actuation with a mass median aerodynamic diameter of less than 4 mcm.

EXAMPLE 5—MESCALINE TROCHE. A batch of 1000 troches are compounded by dispensing 740 grams of PEG 1450 (or PEG 1500+/—PEG 300) (the 740 grams of PEG makes up approximately 60% to 92% total weight) followed by melting the 740 grams of PEG to a maximum temperature of approximately 61 to 72 degrees centigrade on a hot plate. Once the PEG is melted, add powders (citric acid—0.17% to 1.2% by dry weight, stevia—0.4% to 2.5% by dry weight, and acacia gum—0.05% to 2.3% by dry weight, and mix until suspended uniformly. The active ingredient is introduced by adding 3 mg to 100 g of mescaline to yield 200 mg of mescaline per troche. 28 ml of essential oils (e.g., 22 ml of peppermint, 5 ml of menthol (made by dissolving 10 g menthol crystals into 5 ml of peppermint oil and 2 ml of 200 Proof USP grade ethanol) are introduced and the liquid is mixed to uniformity (the concentration of active oil extract is variable to determine total volume of oil to be added). The temperature is maintained between approximately 57 and 65 degrees centigrade. Once the liquid is completely mixed use a sanitary peristaltic pump to deliver 900 mcl per troche (for the dosage form of 0.91 g per troche) and allow mixture to cool at room temperature.

EXAMPLE 6—DMT TRANSDERMAL PATCH. A transdermal patch is prepared utilizing a solution of 20:1 by volume of ethanol to water to dissolve DMT with sufficient stirring. Distilled water is added to achieve a 30 mg per g concentration of DMT in a 3:7 ethanol to water by volume solution. 2.5% by weight of hydroxyethyl cellulose gelling agent is added to this solution with mild agitation for an hour until a smooth gel is created. A 0.05 mm thick contact adhesive layer is formed on a fluorocarbon-diacrylate treated polyester film which comprises the release liner for the system by solution casting an amine resistant silicone medical adhesive onto the polyester film from a solution in trichlorotrifluoroethane. A 0.05 mm thick rate controlling membrane comprised of ethylene-vinyl acetate is pressure laminated to the exposed adhesive. A backing member comprised of a multilaminate of polyethylene, aluminum, polyester and ethylene-vinyl acetate was also provided and the aqueous gel pouched between the backing member and the release liner adhesive/rate controlling membrane on a rotary heat-seal machine at a gel loading of 15 mg/cm2. Sealed pouches in the sizes of 20 cm2 is die cut and immediately pouched to avoid loss of ethanol. This system delivers 9 mcg of DMT per unit dose.

EXAMPLE 7—LSA with sulfobutylether β-cyclodextrin in water air jet nebulizer. Approximatley 12 g of sulfobutyl ether β-cyclodextrin and 63 mg of LSA are placed into a 250 ml volumetric flask. The flask is filled with 3 mM sodium citrate buffer at a pH of 4.5 with 80 mM sodium chloride. The mixture is sonicated in a bath ultrasonic cleaner for 30 minutes at ambient temperature and stirred for 16 hours with a magnetic stirrer. After 16 hours, the flask is sonicated again for 30 minutes. The resulting solution is filtered through a nylon 0.2 mcm syringe filter. Solution after filtration will yield a LSA concentration of 245 mcg per mL. 3 mL of the solution is transferred to a Pari LC Plus Nebulizer with a Pari ProNeb Ultra Air Compressor. This yields a metered dose of 700 mcg of LSA to the pulmonary region of the subject.

EXAMPLE 8—Psilocin Sublingual Thin Film. A feedstock comprising of hydroxypropyl methylcellulose 0 to 15% on a dry weight basis, Eudragit 0 to 15% on a dry weight basis, polyvinyl alcohol 0 to 25% by on a dry weight basis, glycerol 0 to 25% on a dry weight basis and sodium alginate 0 to 15% on a dry weight basis and trehalose 0 to 60% on a dry weight basis and 1.3% psilocin on a dry weight basis. The solution is deposited on a flat Teflon mold and dried in a convection oven at 30 degrees centigrade for 7 hours with a resulting uniform thickness of 0.3 mm of the film. When cut into strips of 3×2 cm, it yields a dose of 2 mg of psilocin to the oral cavity of the subject for sublingual administration.

In one embodiment of the invention, a study of 10 human subjects will have a clinical endpoint of significantly altered state of consciousness (SASC), which is defined as including but not limited to euphoria, enhanced capacity for introspection, and altered psychological functioning with a dream-like hypnagogic experiences, perceptual changes such as illusions, pseudohallucinations, synesthesias, and alterations of thinking and time experience. Subjects will self-administer the DMT pressurised metered-dose inhaler with 1 mg of the active ingredient per actuation up to 10 times. In self administering the product, subjects will be instructed to shake the device vigorously, open mouth, exhale, place inhaler on the mouth, begin inhaling and depress the canister on the inhaler mid breadth with breath held for 5 seconds before exhaling, repeating the steps for each atomization of the product. Subject is instructed to discontinue product use if psychedelic effects are perceived. Subjects will attempt to determine proper dose titration to prevent temporary or permanent negative psychosis while achieving the study end point of SASC and the amount personally required for their sub-psychedelic threshold at that the provided dosing.

In another embodiment, the patient will self-titrate one troche consisting of 200 mg of mescaline which is a 200 mg transmucosal dose to therapeutically achieve SASC.

In another embodiment, the patient will self titrate use of the nasal inhaler of psilocybin to 4 sprays which is a 5.3 mg of psilocybin nasal dose to therapeutically achieve SASC.

In another embodiment, the patient will self-titrate to 3 puffs of the DMT pMDI for a 3 mg pulmonary dose to therapeutically achieve SASC.

In some embodiments, this protocol will reduce the risk of administering combinations of psychedelics for patients who are treatment resistant. For combinations of two active ingredients, a subject will self tirate use of the nasal inhaler of psilocybin to 4 sprays which is a 5.3 mg of psilocybin nasal dose. In a separate session a subject will self tirate to 3 puffs of the DMT pMDI for a 3 mg pulmonary dose. The subject will then be enabled to self dose 4 sprays of the nasal inhaler for 5.3 mg psilocybin absorbed nasally followed in combination by 3 puffs of the pMDI for 3 mg absorbed by the pulmonary region for maximum therapeutic effect of SASC in treatment-resistant subjects and/or scheduled break-through therapy sessions.

In other embodiments, this protocol reduces the risk of administering combinations of psychedelics. For combinations of three active ingredients, subjects will be instructed to administer a fixed dose of one troche consisting of 200 mg of mescaline transmucosally or buccally 30 minutes before administering 4 sprays of psilocybin nasal inhaler delivering 6.5 mg each spray for a total of 26 mg of psilocybin administered nasally which is followed by 3 puffs of the DMT pMDI delivering 3 mg of DMT for pulmonary absorption. This protocol allows for delivery of the psychedelic via parallel routes of administration, transmucosal or buccally, pulmonary and nasally for maximum therapeutic effect of SASC in treatment resistant patients and/or scheduled breakthrough therapy sessions.

In other embodiments, this allows a subject to administer any combination of psychedelic compounds delivered through any combination of routes of administration such as but not limited to oral delivery, nasal delivery, transmucosal or buccal delivery, transdermal delivery, and pulmonary delivery to achieve maximum therapeutic effect of SASC in treatment-resistant patients and/or scheduled breakthrough therapy sessions.

Although the present applications are described in detail above, it will be understood by one of ordinary skill in the art that various modifications can be made without departing from the spirit of the invention. Accordingly, the present application is limited only by the following claims. All cited patents, patent applications and publications referred to in this application are herein incorporated by reference in their entirety. In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure. All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof. As will be appreciated by one of skill in the art, while the present specification may simply use one of the terms “comprise,” “consists,” or “consists essentially of” this is simply a shorthand way of describing all three possibilities, unless otherwise specified or unless the term is used in the claim (in which case the terms will have their normally accepted meanings under claim interpretation). Thus, as the terms are used above, they designate all three possibilities, unless explicitly noted otherwise.

What is claimed is:

1. A metered dosing formulation comprising:
An amount between 3 micrograms to 1.3 g of one or more psychedelic compounds from a class of mushroom-related compounds with any one or more of a preservative, a buffer, a chloride salt, a polymer, a carbohydrate, a solvent, a terpene, a surfactant, a liquified gas, a solvent, and combinations thereof.

2. Formulation of claim 1, wherein said psychedelic compound is selected from said group of mushroom-related compounds comprising psilocybin (O-phosphoryl-4-hydroxy-N,N-dimethyltryptamine, 4-PO-Psilocin, or 4-PO—HO-DMT), psilocin (4-HO-DMT, 4-hydroxy DMT, psilocine, psilocyn, psilotsin), baeocystin, norbaeocystin, salts and isomers thereof, and combinations thereof.

3. Formulation of claim 1, wherein said mushroom-related compounds include synthetic forms or their synthetic analogues thereof.

4. Formulation of claim 1, wherein said formulation comprises a device or formulation that is capable of administering a metered dose between 3 micrograms to 1.3 g of psilocybin (0-phosphoryl-4-hydroxy-N,N-dimethyltryptamine, 4-PO-Psilocin, or 4-PO—HO-DMT), psilocin (4-HO-DMT, 4-hydroxy DMT, psilocine, psilocyn, psilotsin), baeocystin, norbaeocystin, salts and isomers, and combinations thereof.

5. Formulation of claim 1, wherein said formulation further comprises a ratio of at least 0.004 part terpene to 1 part psychedelic compound to less than a ratio of 10 parts terpene to 1 part psychedelic compound.

6. Formulation of claim 1, wherein said formulation is for oral inhalation as delivered by a dry powder inhaler, thermal vaporizer (such as but not limited to an electronic cigarette), air jet nebulizer, vibrating mesh nebulizer, vaporizer, or pressurized metered dose inhaler

7. Formulation of claim 1, wherein said formulation is for nasal inhalation as delivered by nasal spray device containing one or more of water, liquified gas, solvent or thickening agent.

8. Formulation of claim 1, wherein said formulation is for oral administration as delivered by a sublingual film, orally disintegrating tablet, tablet, capsule, lozenge, troche, chewing gum or tincture.

9. Formulation of claim 1, wherein said formulation is for topical administration as delivered by a transdermal patch, topical lotion, or topical spray.

10. A method of use of said metered dosing formulation of claim 1, comprising a single unit dose or an application of a series of single doses until desired effect is reached.

11. A method for treating or mitigating a neurological, physiological, or mental health condition comprising an amount of said formulation from claim 1 applied to a subject thereof.

12. A method for treating or improving neurological or mental health condition comprising an amount of said formulation of claim 1 applied to a subject thereof, wherein said neurological or mental health condition comprises: anxiety, post-traumatic stress disorder, attention deficit disorders, depression, memory loss, dementia, cognitive dysfunction, hearing loss, vision loss, neurologic pain, insomnia, erectile dysfunction, physiological pain or discomfort, or combinations thereof.

13. A metered dosing formulation comprising:
An amount between 720 mcg to 8.4 g of one or more psychedelic compounds from a class of mescaline-related compounds with any one or more of a preservative, a buffer, a chloride salt, a polymer, a carbohydrate, a solvent, a terpene, a surfactant, a liquified gas, a solvent, and combinations thereof.

14. Formulation of claim 13, wherein said mescaline-related compounds comprises mescaline (3,4,5-trimethoxyphenethylamine) or salts and isomers thereof (such as mescaline hydrochloride or mescaline fumarate); extracts of peyote (Lophophora williamsii), San Pedro (Echinopsis pachanoi), and Peruvian torch (Echinopsis/Trichocereus peruviana) cactus; and combinations thereof.

15. Formulation of claim 13. wherein said mescaline-related compounds include synthetic forms or their synthetic analogues thereof.

16. Formulation of claim 13, wherein said formulation comprises a dose between 3 micrograms to 1.3 g of mescaline (3,4,5-trimethoxyphenethylamine) or salts and isomers thereof (such as mescaline hydrochloride or mescaline fumarate); extracts of peyote (Lophophora williamsii), San Pedro (Echinopsis pachanoi), and Peruvian torch (Echinopsis/Trichocereus peruviana) cactus; and combinations thereof.

17. Formulation of claim 13, wherein said formulation further comprises a ratio of at least 0.004 part terpene to 1 part psychedelic compound to less than a ratio of 10 parts terpene to 1 part psychedelic compound.

18. Formulation of claim 13, wherein said formulation is for oral inhalation as delivered by a dry powder inhaler, thermal vaporizer (such as but not limited to an electronic cigarette), air jet nebulizer, vibrating mesh nebulizer, vaporizer, or pressurized metered dose inhaler

19. Formulation of claim 13, wherein said formulation is for nasal inhalation as delivered by nasal spray device containing one or more of water, liquified gas, solvent or thickening agent.

20. Formulation of claim 13, wherein said formulation is for oral administration as delivered by a sublingual film, tablet, capsule, lozenge, troche, chewing gum or tincture

21. Formulation of claim 13, wherein said formulation is for topical administration as delivered by a transdermal patch, topical lotion, or topical spray.

22. A method of use of said metered dosing formulation of claim 13, comprising a single unit dose or an application of a series of single doses until desired effect is reached.

23. A method for treating or mitigating a neurological, physiological or mental health condition comprising an amount of said formulation from claim 13 applied to a subject thereof.

24. A method for treating or improving neurological or mental health condition comprising an amount of said formulation of claim 13 applied to a subject thereof, wherein said neurological or mental health condition comprises: anxiety, post-traumatic stress disorder, attention deficit disorders, depression, memory loss, insomnia, erectile dysfunction, dementia, cognitive dysfunction, hearing loss, vision loss, neurologic pain, physiological pain or discomfort, or combinations thereof.

25. A metered dosing formulation comprising:
An amount between 75 mcg to 830 mg of one or more psychedelic compounds from a class of DMT-related compounds with any one or more of a preservative, a buffer, a chloride salt, a polymer, a carbohydrate, a solvent, a terpene, a surfactant, a liquified gas, a solvent, and combinations thereof.

26. Formulation of claim 25, wherein said psychedelic compound is selected from the group comprising N,N-dimethyltryptamine, N,N-DMT, extracts of DMT-related compounds, and combinations thereof.

27. The formulation of claim 25, further comprising a monoamine oxidase inhibitor (MAOI).

28. Formulation of claim 25, wherein said DMT-related compounds includes synthetic forms or their synthetic analogues thereof.

29. Formulation of claim 25, wherein said formulation comprises a dose between 3 micrograms to 1.3 g of N,N-dimethyltryptamine, N,N-DMT, extracts of DMT-related compounds, and combinations thereof.

30. Formulation of claim 25, wherein said formulation further comprises a ratio of at least 0.004 part terpene to 1 part psychedelic compound to less than a ratio of 10 parts terpene to 1 part psychedelic compound.

31. Formulation of claim 25, wherein said formulation is for oral inhalation as delivered by a dry powder inhaler, thermal vaporizer (such as but not limited to an electronic cigarette), air jet nebulizer, vibrating mesh nebulizer, vaporizer, or pressurized metered dose inhaler

32. Formulation of claim 25, wherein said formulation is for nasal inhalation as delivered by nasal spray device containing one or more of water, liquified gas, solvent or thickening agent.

33. Formulation of claim 25, wherein said formulation is for oral administration as delivered by a sublingual film, tablet, capsule, lozenge, troche, chewing gum or tincture

34. Formulation of claim 25, wherein said formulation is for topical administration as delivered by a transdermal patch, topical lotion, or topical spray.

35. A method of use of said metered dosing formulation of claim 25, comprising a single unit dose or an application of a series of single doses until desired effect is reached.

36. A method for treating or mitigating a neurological, physiological or mental health condition comprising an amount of said formulation from claim 25 applied to a subject thereof.

37. A method for treating or improving neurological or mental health condition comprising an amount of said formulation of claim 25 applied to a subject thereof, wherein said neurological or mental health condition comprises: anxiety, post-traumatic stress disorder, attention deficit disorders, depression, memory loss, insomnia, erectile dysfunction, dementia, cognitive dysfunction, hearing loss, vision loss, neurologic pain, physiological pain or discomfort, or combinations thereof.

38. A metered dosing formulation comprising:
An amount between 150 mcg to 5 g of one or more psychedelic compounds from a class of LSA- and Ibogaine-related compounds with any one or more of a preservative, a buffer, a chloride salt, a polymer, a carbohydrate, a solvent, a terpene, a surfactant, a liquified gas, a solvent, and combinations thereof.

39. Formulation of claim 38, wherein said psychedelic compound is selected from the group comprising an amount of D-lysergic acid amide (LSA), also-known as Ergine and d-lysergamide or salts and isomers thereof; extracts of plants or seeds of the convolvulaceae or morning glory family; Ibogaine; extracts of plants of the Apocynacea family, such as Tabernanthe iboga (or “iboga”) Voacanga african, or combinations thereof.

40. Formulation of claim 38, wherein said LSA-related compounds or Ibogaine-related compounds includes synthetic forms or their synthetic analogues thereof.

41. Formulation of claim 38, wherein said formulation comprises a dose between 3 micrograms to 1.3 g of D-lysergic acid amide (LSA), also-known as Ergine and d-lysergamide or salts and isomers thereof; extracts of plants or seeds of the convolvulaceae or morning glory family; Ibogaine; extracts of plants of the Apocynacea family, such as Tabernanthe iboga (or “iboga”) Voacanga african, or combinations thereof.

42. Formulation of claim 38, wherein said formulation further comprises a ratio of at least 0.004 part terpene to 1 part psychedelic compound to less than a ratio of 10 parts terpene to 1 part psychedelic compound.

43. Formulation of claim 38, wherein said formulation is for oral inhalation as delivered by a dry powder inhaler, thermal vaporizer (such as but not limited to an electronic cigarette), air jet nebulizer, vibrating mesh nebulizer, vaporizer, or pressurized metered dose inhaler

44. Formulation of claim 38, wherein said formulation is for nasal inhalation as delivered by nasal spray device containing one or more of water, liquified gas, solvent or thickening agent.

45. Formulation of claim 38, wherein said formulation is for oral administration as delivered by a sublingual film, tablet, capsule, lozenge, troche, chewing gum or tincture

46. Formulation of claim 38, wherein said formulation is for topical administration as delivered by a transdermal patch, topical lotion, or topical spray.

47. A method of use of said metered dosing formulation of claim 38, comprising a single unit dose or an application of a series of single doses until desired effect is reached.

48. A method for treating or mitigating a neurological, physiological or mental health condition comprising an amount of said formulation from claim 38 applied to a subject thereof.

49. A method for treating or improving neurological or mental health condition comprising an amount of said formulation of claim 38 applied to a subject thereof, wherein said neurological or mental health condition comprises: anxiety, post-traumatic stress disorder, attention deficit disorders, depression, memory loss, insomnia, erectile dysfunction, dementia, cognitive dysfunction, hearing loss, vision loss, neurologic pain, physiological pain or discomfort, or combinations thereof.

50. A metered dosing formulation comprising:
An amount between 10 mcg to 500 mg of one or more psychedelic compounds from a class of psychedelic synthetics or their synthetic analogues with any one or more of a preservative, a buffer, a chloride salt, a polymer, a carbohydrate, a solvent, a terpene, a surfactant, a liquified gas, a solvent, and combinations thereof.

51. Formulation of claim 50, wherein said psychedelic compound is a synthetic or analog of naturally occurring psychedelic tryptamine, psilocybin, psilocin, baeocystin, norbaeocystin, or salts thereof, extracts of psilocybin mushrooms or synthetic forms thereof, mescaline, 3,4,5-trimethoxyphenethylamine, extracts of peyote or synthetic forms thereof, N,N-dimethyltryptamine, N,N-DMT, extracts of DMT-related compounds or synthetic forms thereof, LSA/Ergine, other plant or fungal derived psychedelic compounds including but not limited to ibogaine, or combinations thereof.

52. Formulation of claim 50, wherein said formulation comprises a dose between 3 micrograms to 1.3 g of a synthetic or analog of naturally occurring psychedelic tryptamine, psilocybin, psilocin, baeocystin, norbaeocystin, or salts thereof, extracts of psilocybin mushrooms or synthetic forms thereof, mescaline, 3,4,5-trimethoxyphenethylamine, extracts of peyote or synthetic forms thereof, N,N-dimethyltryptamine, N,N-DMT, extracts of DMT-related compounds or synthetic forms thereof, LSA/Ergine, other plant or fungal derived psychedelic compounds including but not limited to ibogaine, or combinations thereof.

53. Formulation of claim 50, wherein said formulation further comprises a ratio of at least 0.004 part terpene to 1 part psychedelic compound to less than a ratio of 10 parts terpene to 1 part psychedelic compound.

54. Formulation of claim 50, wherein said formulation is for oral inhalation as delivered by a dry powder inhaler, thermal vaporizer (such as but not limited to an electronic cigarette), air jet nebulizer, vibrating mesh nebulizer, vaporizer, or pressurized metered dose inhaler

55. Formulation of claim 50, wherein said formulation is for nasal inhalation as delivered by nasal spray device containing one or more of water, liquified gas, solvent or thickening agent.

56. Formulation of claim 50, wherein said formulation is for oral administration as delivered by a sublingual film, tablet, capsule, lozenge, troche, chewing gum or tincture

57. Formulation of claim 50, wherein said formulation is for topical administration as delivered by a transdermal patch, topical lotion, or topical spray.

58. A method of use of said metered dosing formulation of claim 50, comprising a single unit dose or an application of a series of single doses until desired effect is reached.

59. A method for treating or mitigating a neurological, physiological or mental health condition comprising an amount of said formulation from claim 50 applied to a subject thereof.

60. A method for treating or improving neurological or mental health condition comprising an amount of said formulation of claim 50 applied to a subject thereof, wherein said neurological or mental health condition comprises: anxiety, post-traumatic stress disorder, attention deficit disorders, depression, memory loss, insomnia, erectile dysfunction, dementia, cognitive dysfunction, hearing loss, vision loss, neurologic pain, physiological pain or discomfort, or combinations thereof.

61. A metered dose nasal spray comprising:
a metered dosing formulation further comprising an amount of at least one or more psychedelic compound(s) said psychedelic compound selected from the group comprising mushroom-related compounds, mescaline-related compounds, DMT-related compounds, LSA-related compounds, Ibogaine-related compounds, or synthetic compounds or their synthetic analogs thereof;
a solvent; and
a nasal spray device to administer said formulation via the nasal passages.

62. The nasal spray from claim 61 further comprising of at least two of:
a terpene;
an emulsifying agent;
a preservative;
a thickening agent;
a monoamine oxidase inhibitor;
a solvent;
a liquified gas;
or combinations thereof.

63. The metered dose spray of claim 61, wherein said canister further comprises a metered dose valve that is configured to dispense between 25 and 200 microliters of formulation per actuation.

64. A formulation for metered dose inhalation comprising:
an amount of at least one of psilocybin, psilocin, or extract of psilocybe mushrooms, wherein the amount of at least one of psilocybin, psilocin, or extract of psilocybe mushrooms is a dose between 3 micrograms and 5 mg; an amount of propellant suitable for metered dose inhalation application to a human subject, wherein the propellant comprises at least one HFA;
a solvent.

65. A formulation for metered dose inhalation comprising:
an amount of at least one of mescaline (3,4,5-trimethoxyphenethylamine) or salts and isomers thereof (such as mescaline hydrochloride or mescaline fumarate); extracts of peyote (Lophophora wilhamsii), San Pedro (Echinopsis pachanoi), and Peruvian torch (Echinopsis/Trichocereus peruviana) cactus; and combinations thereof;
an amount of propellant suitable for metered dose inhalation application to a human subject, wherein the propellant comprises at least one HFA; and
a solvent and/or surfactant.

66. A formulation for metered dose inhalation, said formulation comprising:
an amount of N,N-dimethyltryptamine, N,N-DMT, extracts of DMT-related compounds, and combinations thereof.
an amount of propellant suitable for metered dose inhalation application to a human subject, wherein the propellant comprises at least one HFA; and
a solvent and/or surfactant and/or MAOI.
